(19) United States
(12) Patent Application Publication
(10) Pub. No.: US 2026/0179777 A1
(43) Pub. Date: Jun. 25, 2026

(54) System And Method For Assessing Hypotensive Risk Using Machine Learning Models To Analyze Health Data And Output A Hypotensive Risk Index

(71) Applicant: REGENTS OF THE UNIVERSITY OF MICHIGAN, Ann Arbor, MI (US)

(72) Inventors: Kevin R. Ward, Superior Township, MI (US); Mohamad Hakam Tiba, South Lyon, MI (US); Brandon C. Cummings, White Lake, MI (US); Daniel Ehrmann, Ann Arbor, MI (US)

(21) Appl. No.: 19/418,140

(22) Filed: Dec. 12, 2025

Related U.S. Application Data
(60) Provisional application No. 63/736,367, filed on Dec. 19, 2024.

(51) Int. Cl. G16H 50/30

(52) U.S. Cl. CPC G16H 50/30

(57) ABSTRACT

A computing system processes digital health data to determine a patient-specific hypotension threshold, quantify hypotension, derive vascular performance metrics, and output a Hypotensive Risk Index (HRI) communicated to clinicians. A method involves processing health data to determine a hypotension threshold, quantify hypotension, derive related metrics, and output an HRI communicated to clinicians. A computer-readable medium contains instructions for processing health data to determine a hypotension threshold, quantify hypotension, derive metrics, and output an HRI communicated to clinicians.

### CROSS-REFERENCE TO RELATED APPLICATIONS

The present application claims priority to U.S. Provisional Application No. 63/736,367, entitled System And Method For Assessing Hypotensive Risk Using Machine Learning Models To Analyze Health Data And Output A Hypotensive Risk Index, filed on Dec. 19, 2024, the entirety of which is hereby incorporated by reference.

### FIELD OF THE INVENTION

The present aspects relate to health monitoring systems, and more particularly, to systems and methods for assessing and predicting hypotensive risk using machine learning models, for example, by determining an adaptive threshold of hypotension for a patient.

### BACKGROUND

The development of hypotension can result in many downstream health complications, including but not limited to multiple organ failure, neurologic injury, and death. These complications are typically due to critical reductions in organ blood flow and oxygen delivery. Blood pressure (BP) and its components (systolic, diastolic, and mean arterial BP) are commonly used as single point-in-time metrics to assess hypotension and its associated risks. However, the duration and intensity of hypotensive episodes have been shown to be key indicators of future health complications. Hypotension and its management in clinical settings, such as operating rooms or intensive care units, represent a significant challenge. Traditional methods of monitoring blood pressure, whether invasive or non-invasive, offer snapshots of a patient's hemodynamic status but often fail to provide comprehensive insights into the cumulative effects of hypotensive episodes over time. Moreover, these methods do not typically take into account the individual patient's history or the nuances of their current condition, which can significantly impact the interpretation of blood pressure readings. The lack of analytical tools to quantify the cumulative exposure to hypotension and to predict potential complications or the need for therapeutic interventions exacerbates the problem. Current systems may allow for the trending of hypotensive episodes but do not offer quantitative analysis or predictive capabilities concerning the patient's risk of developing complications. The complexity of managing hypotension is further compounded by the relative nature of blood pressure thresholds, which can vary from one patient to another based on their medical history and baseline blood pressure levels. This variability introduces challenges in setting standardized criteria for identifying and responding to hypotensive events. Given these limitations, there are therefore opportunities for improved platforms and technologies for solving the identified conventional problems.

### BRIEF SUMMARY OF THE INVENTION

In one aspect, a computing system includes: (1) a processor and (2) a memory that includes computer-executable instructions that, when executed, cause the computing system to (a) receive digital health data for a patient from one or more sources; (b) determine an adaptive threshold of hypotension for the patient using one or more trained machine learning models; (c) quantify the duration and intensity of hypotension using either an adaptive or static threshold into one or several quantities of interest; (d) derive additional quantities of interest related to vascular performance and/or associated clinical risk; (e) integrate the quantities of interest into one or more trained machine learning models; (f) output a Hypotensive Risk Index (HRI) score based on the quantities of interest provided to the trained machine learning model(s); and (g) communicate the HRI to a clinician or other end user via a remote device.

In another aspect, a computer-implemented method includes: (1) receiving, via one or more processors, digital health data for a patient from one or more sources; (2) determining, via one or more processors, an adaptive threshold of hypotension for the patient using one or more trained machine learning models; (3) quantifying, via one or more processors, the duration and intensity of hypotension using either an adaptive or static threshold into one or several quantities of interest; (4) deriving, via one or more processors, additional quantities of interest related to vascular performance and/or associated clinical risk; (5) integrating, via one or more processors, the quantities of interest into one or more trained machine learning models; (6) outputting, via one or more processors, a Hypotensive Risk Index (HRI) score based on the quantities of interest provided to the trained machine learning model(s); and (7) communicating, via one or more processors, the HRI to a clinician or other end user via a remote device.

In yet another aspect, a computer-readable medium includes instructions that when executed cause a computer to perform steps including: (1) receiving digital health data for a patient from one or more sources; (2) determining an adaptive threshold of hypotension for the patient using one or more trained machine learning models; (3) quantifying the duration and intensity of hypotension using either an adaptive or static threshold into one or several quantities of interest; (4) deriving additional quantities of interest related to vascular performance and/or associated clinical risk; (5) integrating the quantities of interest into one or more trained machine learning models; (6) outputting a Hypotensive Risk Index (HRI) score based on the quantities of interest provided to the trained machine learning model(s); and (7) communicating the HRI to a clinician or other end user via a remote device.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The patent or application file contains at least one drawing executed in color. Copies of this patent or patent application publication with color drawings will be provided by the Office upon request and payment of the necessary fee.

The figures described below depict various aspects of the system and methods disclosed herein. It should be understood that each figure depicts an embodiment of a particular aspect of the disclosed system and methods, and that each of the figures is intended to accord with a possible embodiment thereof.

There are shown in the drawings arrangements which are presently discussed, it being understood, however, that the present embodiments are not limited to the precise arrangements and instrumentalities shown, wherein:

FIG. 1 depicts a computing environment for quantifying prolonged hypotension and assessing associated clinical risk, according to some aspects.

FIG. 2 depicts a computer-implemented method for quantifying and assessing hypotensive risk in patients, according to some aspects.

FIG. 3 depicts a flow diagram of a computer-implemented method for quantifying and integrating duration and intensity of hypotension into a Hypotensive Risk Index (HRI), according to some aspects.

FIG. 4A depicts a graph of hypothetical patient's SBP over time along with an example threshold of 90 mmHg, according to some aspects.

FIG. 4B depicts a graph of hypothetical patient's BP deficit over time, corresponding to FIG. 4A, according to some aspects.

FIG. 4C depicts a graph of hypothetical patient's hypotension over time, corresponding to FIG. 4A and FIG. 4B, according to some aspects.

FIG. 5 depicts a plot of an example Shapley value plotted against a feature value, in particular temperature, according to some aspects.

### DETAILED DESCRIPTION

Given the complexity and critical nature of managing hypotension in clinical settings, the disclosed systems and methods introduce a transformative approach to quantifying prolonged hypotension and its associated clinical risks. This approach leverages advanced computational techniques, including machine learning, to provide continuous, real-time analysis of hypotension episodes and their potential impact on patient outcomes. By integrating a wide array of data sources, from electronic health records (EHR) to various monitoring sensors, this methodology offers a comprehensive view of a patient's vascular performance and associated risks, thereby enabling more informed clinical decision-making.

One of the significant challenges in current clinical practice is the lack of tools for quantitatively assessing the duration and intensity of hypotension episodes and their cumulative effect on patient health. Traditional methods of blood pressure monitoring provide only snapshots of a patient's condition, without capturing the dynamic nature of hypotension and its implications over time. The disclosed systems and methods address this gap by introducing the concept of hypotension deficit and debt, which quantitatively measure the extent and duration of hypotension episodes relative to patient-specific thresholds. This innovative approach allows for a more nuanced understanding of hypotension's impact, facilitating early intervention and personalized care strategies.

The integration of machine learning models represents another cornerstone of the disclosed methodology. By analyzing a wide range of data inputs, including vital signs, laboratory values, and patient history, these models can predict the likelihood of various complications associated with hypotension, such as organ failure, sepsis, pneumonia, neurologic injury, the post intensive care syndrome, death, and others. This predictive capability is crucial for proactive patient management, enabling clinicians to anticipate and mitigate potential adverse outcomes. Moreover, the use of machine learning allows for the continuous refinement of predictive models based on new data and outcomes, ensuring that the methodology remains at the cutting edge of clinical practice.

The detailed description of the disclosed systems and methods will delve into several key areas, including data acquisition, noise reduction and cleaning, blood pressure preprocessing, and the derivation of additional metrics. Each of these components plays a vital role in the overall methodology, contributing to the accuracy and reliability of the hypotensive risk index (HRI). The HRI, in turn, serves as a valuable tool for clinicians, providing a quantifiable measure of a patient's risk of hypotension-related complications. By offering a comprehensive and dynamic assessment of hypotension, the disclosed systems and methods represent a significant advancement in patient monitoring and care.

Furthermore, the detailed description will explore the technical underpinnings of the methodology, including the algorithms and models used for threshold determination, deficit and debt quantification, and the construction of the HRI. These technical aspects are critical for understanding how the disclosed systems and methods achieve their predictive and analytical capabilities. Additionally, the description will address the practical implementation of the methodology, including the communication of HRI scores to clinicians and the integration of the systems and methods into existing clinical workflows. Through this comprehensive exploration, the detailed description will provide a thorough understanding of the disclosed systems and methods, highlighting their potential to transform the management of hypotension in clinical settings.

The present techniques focus on enhancing the management and assessment of hypotension in patients through the utilization of advanced computing systems and machine learning models. These techniques aim to address the critical challenge of quantifying the duration and intensity of hypotension episodes and integrating these measurements with other relevant clinical data to assess the risk of associated complications. By doing so, the present techniques offer significant improvements in processing efficiency, network usage, and memory utilization within computing environments dedicated to healthcare monitoring and analysis.

One of the primary improvements offered by the present techniques is in processing efficiency. The computing system is designed to receive and process digital health data, including blood pressure data from various sources such as continuous and intermittent noninvasive devices, as well as invasive arterial blood catheters. This data is then standardized and, if necessary, interpolated to fill in missing segments. Such preprocessing steps are crucial for ensuring that the data fed into the machine learning models is of high quality and uniformity, thereby enhancing the efficiency of the subsequent analysis processes. The application of a multiple imputation strategy using a Bayesian ridge regression framework further exemplifies the sophisticated data handling capabilities of the present techniques, enabling the system to impute missing values based on the patient's current condition effectively.

Another significant improvement is in network usage. The present techniques are designed to communicate the Hypotensive Risk Index (HRI) score, a critical output of the system's analysis, to clinicians or other end users via remote devices. This communication process is optimized to ensure that the data transmission is both secure and efficient, minimizing the bandwidth required for transmitting potentially large datasets or complex analytical results. By leveraging advanced data compression and encryption methods, the present techniques ensure that the critical information reaches the intended recipients promptly and securely, facilitating timely clinical decision-making.

Memory usage is also optimized in the present techniques. The system's ability to preprocess and standardize incoming data, as well as to apply sophisticated imputation strategies, allows for the efficient utilization of memory resources. By ensuring that only relevant and high-quality data is stored and processed, the present techniques minimize the unnecessary consumption of memory, thereby enhancing the overall performance of the computing system. This is particularly important in healthcare settings where large volumes of data are generated continuously, and the efficient management of computing resources is critical.

In summary, the present techniques introduce a comprehensive approach to managing and analyzing hypotension in patients, leveraging advanced computing systems and machine learning models. By focusing on improvements in processing efficiency, network usage, and memory utilization, these techniques offer a robust solution for healthcare providers to assess the risk of hypotension-related complications more effectively. Through the integration of various data sources and the application of sophisticated data processing and analysis methods, the present techniques represent a significant advancement in the field of health monitoring and risk assessment.

### Exemplary Computing Environment

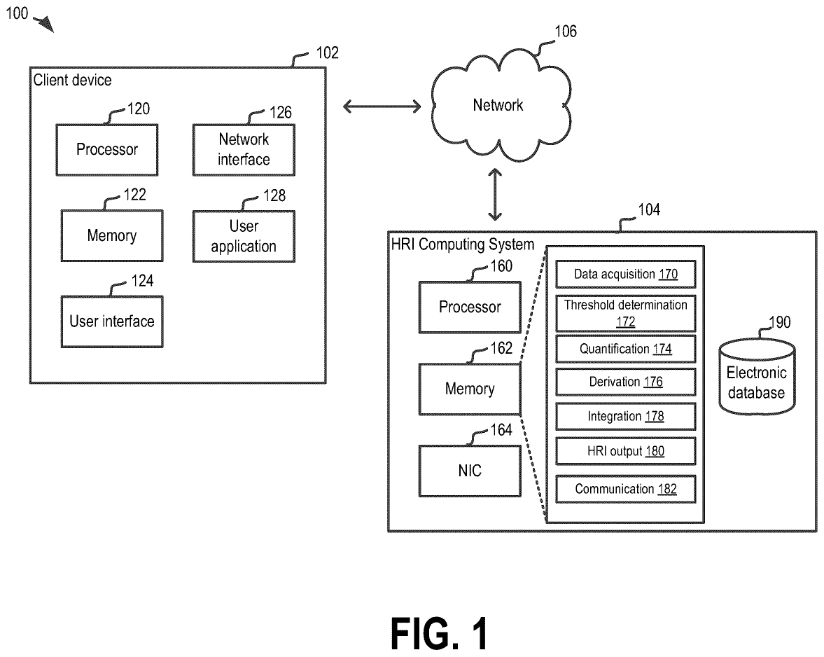

FIG. 1 depicts a computing environment 100 for quantifying prolonged hypotension and assessing associated clinical risk. The computing environment 100 includes a client device 102 and a HRI computing system 104 communicatively coupled by an electronic network 106. The client device 102 may include a processor 120, a memory 122, a user interface 124, a network interface controller (NIC) 126 and a user application 128. The client device 102 is generally configured to receive HRI scores and reports, and to display that information to end users.

The HRI computing system 104 may include a processor 160, a memory 162, a user application 164 and a network interface 166. The HRI computing system 104 may also include an electronic database 190. The HRI computing system 104 may be designed to receive digital health data for a patient from various sources, determine an adaptive threshold of hypotension using one or more trained machine learning models, quantify the duration and intensity of hypotension, derive additional quantities of interest related to vascular performance and/or associated clinical risk, integrate these quantities into trained machine learning models, output a Hypotensive Risk Index (HRI) score, and communicate the HRI to clinicians or other end users via remote devices (e.g., the client device 102).

The processor 120 and the processor 160 may include any number of processors and/or processor types, such as central processing units (CPUs), graphics processing units (GPUs), field-programmable gate arrays (FPGAs), application-specific integrated circuits (ASICs), digital signal processors (DSPs), neural processing units, RISC-V processors, coprocessors, specialized processors/accelerators for artificial intelligence (AI) or machine learning (ML)-specific applications, one or more microcontrollers, and the like. Generally, the processors are configured to execute software instructions stored in the memory.

The memory 122 and the memory 162 may include volatile and/or non-volatile fixed and/or removable memory, such as read-only memory (ROM), electronic programmable read-only memory (EPROM), random access memory (RAM), erasable electronic programmable read-only memory (EEPROM), and/or other hard drives, flash memory, solid-state drives, optical drives, MicroSD cards, and others. The memory 122 and the memory 162 have stored thereon one or more respective sets of computer-executable instructions.

The memory 162 instructions may include a data acquisition module 170 for receiving digital health data, a threshold determination module 172 for determining an adaptive threshold of hypotension, a quantification module 174 for quantifying the duration and intensity of hypotension, an additional metrics derivation module 176 for deriving additional quantities of interest, an integration module 178 for integrating the quantities into machine learning models, an HRI output module 180 for outputting the HRI score, and a communication module 182 for communicating the HRI to clinicians or other end users.

The data acquisition module 170 may include instructions that, when executed, cause the HRI computing system 104 to receive a nested data structure of electronic records that include digital health data of a patient. The data may be time series data, in some aspects. This module is designed to interface with various data sources, such as electronic health records (EHR), wearable devices, and monitoring equipment, to collect comprehensive health data including but not limited to blood pressure readings, heart rate, and other vital signs. The module is capable of handling data in various formats and from multiple sources simultaneously, ensuring a seamless aggregation of patient health information.

The threshold determination module 172 may include instructions that, when executed, enable the HRI computing system 104 to apply one or more trained machine learning models to determine an adaptive threshold of hypotension specific to the patient. This module analyzes the received health data in conjunction with historical patient data and population health statistics to establish a personalized hypotension threshold that accounts for individual variability in blood pressure norms and responses.

In general, the “hypotension threshold” or “threshold” represents the value below which the patient is considered to be experiencing hypotension. This is often treated as a static value: for example, an SBP of 90 mmHg or below is considered a common threshold for hypotension, with lower values indicating higher severity. However, the precise pressure at which a given patient begins to experience an elevated risk of future complications depends upon many individualized factors, including but not limited to their physiologic status, compensatory reserve, medical history and comorbidities, and current level of care. Thus, the threshold determination module 172 may include multiple sets of instructions for computing the threshold value according to one or more of the following methods:
(i) mathematically derived thresholds based on current EHR, waveform, and other clinical data in combination with historical data from similar patients. This may be considered a mechanism for hypotension threshold optimization.
(ii) clinician-specified thresholds. For example, a clinician may choose to override the default threshold from (i) to decrease or increase the threshold based on the clinical presentation of the patient.
(iii) a preset, static threshold value for all patients in a given ward, service, or hospital. For example, a threshold of 90 mmHg may be set by the system for all patients.

One embodiment of a method for (i) mathematically deriving a threshold value uses an analog of shock index (SI). SI is a widely used derived quantity used by clinicians for detecting hypovolemic shock, and is defined as HR divided by SBP. In this embodiment, trends in HR and SBP are correlated over time to estimate the cardiovascular status of the patient at a given pressure. All else being equal, an increase in HR is typically followed by a corresponding increase in SBP. If SBP decreases despite an increase in HR, it indicates the patient may not have the compensatory ability to raise their SBP further. The pressure at which an increase in HR does not correspond to an increase in SBP can then be used as the hypotension threshold.

In the case that BP is being derived from an arterial line, a second embodiment may use an estimate of Stroke Volume (SV) to further inform the threshold determination. Defined as the volume of blood that is pumped through the ventricle with each beat of the heart, SV has been shown to be reliably estimated from the arterial line signal. This can in turn be combined with HR to estimate cardiac output (CO), the volume of blood pumped through the heart per unit time. If HR is increasing, but BP and CO are decreasing, this indicates the patient may be struggling to maintain their physiologic status. The pressure at which this occurs can thus be used as the hypotension threshold.

In another aspect, the threshold determination module 172 may introduce oxygenation status to the above metric, including but not limited to SpO2, SvO2, or blood gas readings. As much of the organ damage and other deleterious effects caused by hypotensive episodes is due to a lack of oxygen reaching the tissues, identifying the BP at which oxygen delivery becomes impacted may be used to identify harmful levels of hypotension.

In yet another aspect, the threshold determination module 172 may use a trained machine learning model to predict the optimal hypotension threshold. In this embodiment, one or more of the above quantities of interest may be combined with laboratory, vital sign, diagnostic, or other clinical data identified above into a model which has, using retrospective data, been trained to identify the BP threshold at which the patient experiences an increased risk of poor clinical outcomes.

The quantification module 174 may include instructions that, when executed, facilitate the HRI computing system 104 in quantifying the duration and intensity of hypotension episodes using either an adaptive or static threshold. This module calculates various metrics such as the total time spent below the hypotension threshold and the depth of each hypotensive episode, providing a detailed view of the patient's exposure to hypotension.

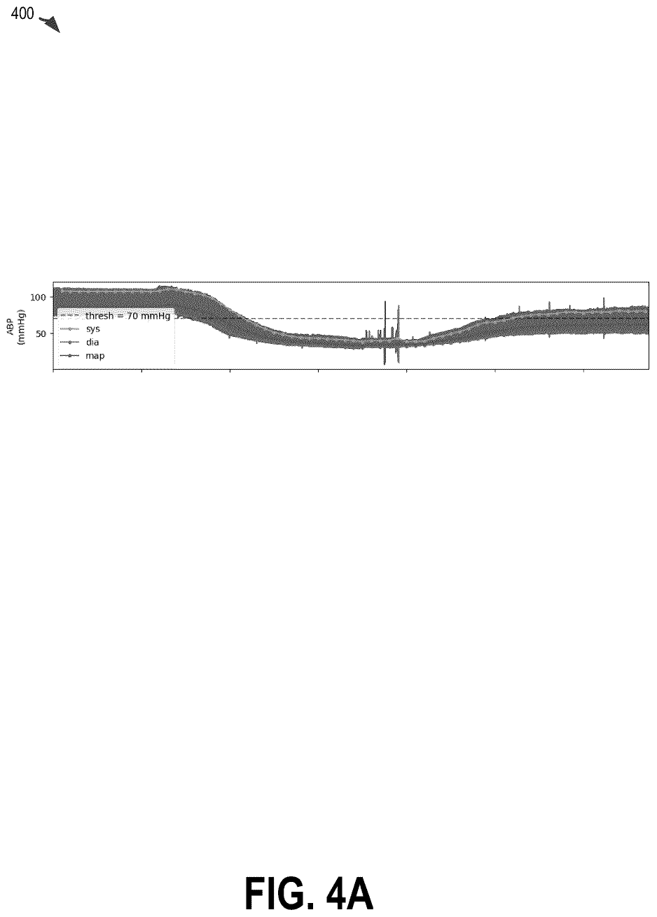

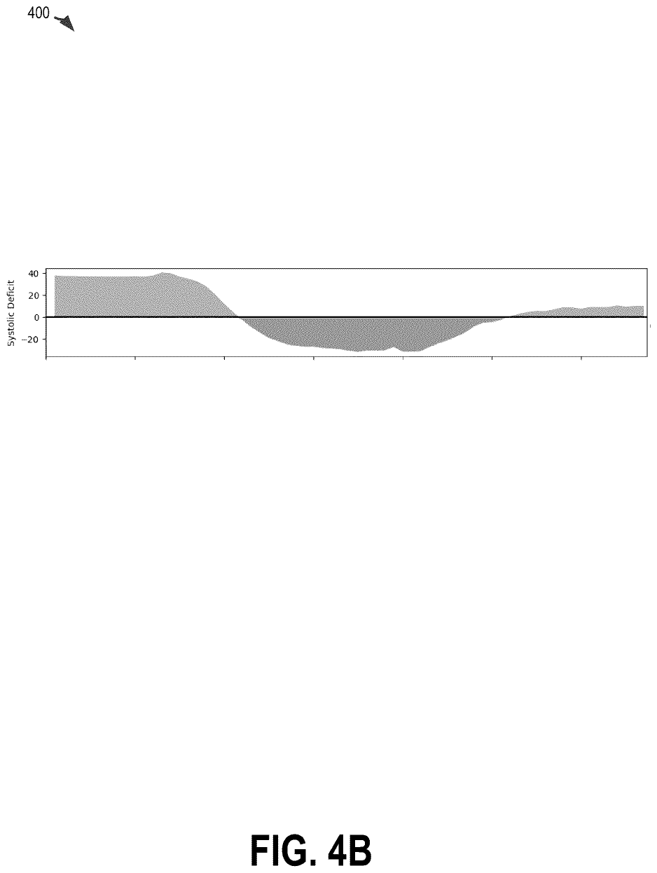

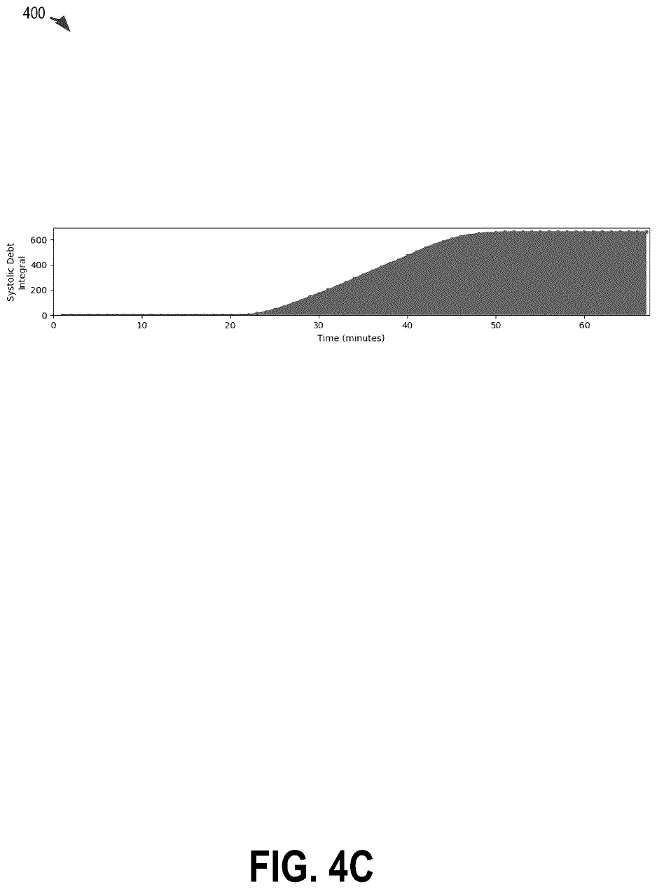

The quantification module 174 may perform deficit and debt quantification, in some aspects, as depicted in FIGS. 4A-4C, below.

The additional metrics derivation module 176 may include instructions that, when executed, cause the HRI computing system 104 to derive additional quantities of interest related to vascular performance and associated clinical risk. This module leverages advanced algorithms to analyze the aggregated health data and extract insights into vascular health, such as variability in blood pressure, pulse pressure, and other hemodynamic parameters that may indicate underlying vascular dysfunction or risk.

For example, the additional metrics derivation module 176 may derive additional metrics. In addition to the acquisition of blood pressure, many other components may be measured in certain embodiments of the methodology. These may include:
(i) Tabular parameters: laboratory values, vital signs, diagnoses, comorbidities, medications, hospital unit transfers, level of care, or other physiological or contextual information relevant to the model. This data is principally retrieved from the electronic health record, but may also be provided by a third-party data aggregation platform.
(ii) Continuous or near-continuous signals (waveforms): electrocardiogram (ECG), photoplethysmography (PPG), intracranial pressure (ICP), or other physiologic signals. This data is principally retrieved from bedside monitors and third party data aggregation platforms.
(iii) Derived quantities of interest: quantities calculated either within the method itself, or provided by third party platforms. These include quantities of interest derived from individual features (e.g. heart rate variability [HRV] measured from ECG, SpO2 from PPG) or as combinations of features (e.g. shock index as a combination of HR and SBP). Derived quantities may include shock index (SI), pulse pressure (PP), etc. as well as other listed features. Any of these features may be derived data, yet it will be appreciated that these features may be determined by separate sources of data, such as sources of data for SpO2, cardiac output, HRV, etc. Thus, in various examples, the techniques herein may receive blood pressure data from continuous noninvasive blood pressure monitoring devices or receive blood pressure derived data, such as the aforementioned data values.

The integration module 178 may include instructions that, when executed, enable the HRI computing system 104 to integrate the derived quantities of interest into one or more trained machine learning models. This module synthesizes the collected data and derived metrics to enhance the predictive models' accuracy in assessing hypotensive risk, ensuring that the models are continuously updated with the latest patient data and health insights.

The HRI output module 180 may include instructions that, when executed, cause the HRI computing system 104 to output a Hypotensive Risk Index (HRI) score based on the quantities of interest provided to the trained machine learning model(s). This module calculates the HRI score, a composite metric that quantifies the patient's risk of hypotension-related complications, enabling clinicians to make informed decisions regarding patient care.

The communication module 182 may include instructions that, when executed, enable the HRI computing system 104 to communicate the HRI score to clinicians or other end users via a remote device. This module ensures the secure and efficient transmission of the HRI score and any relevant health data to healthcare providers, facilitating real-time monitoring and timely intervention for patients at risk of hypotension.

The NIC 166 includes any suitable network interface controller(s), such as wired/wireless controllers (e.g., Ethernet controllers), and facilitates bidirectional/multiplexed networking over the network between the computing environment 100 and various data sources, remote devices, and other components of the computing environment 100. The network 106 may be a single communication network or may include multiple communication networks of one or more types (e.g., one or more wired and/or wireless local area networks (LANs), and/or one or more wired and/or wireless wide area networks (WANs) such as the Internet).

In operation, the computing environment 100 receives digital health data for a patient from sources such as electronic health records (EHR), sensors (including but not limited to pressure transducers, photoplethysmography, or electrocardiogram sensors), or other sources. It then determines an adaptive threshold of hypotension for the patient using one or more trained machine learning models. The system quantifies the duration and intensity of hypotension using either an adaptive or static threshold into one or several quantities of interest. It derives additional quantities of interest related to vascular performance and/or associated clinical risk. These quantities of interest are integrated into one or more trained machine learning models. The system outputs an HRI score based on the quantities of interest provided to the trained machine learning model(s) and communicates the HRI to a clinician or other end user via a remote device. This process enables healthcare providers to assess the risk of hypotension-related complications in real-time, facilitating timely and informed clinical decisions.

### Exemplary Computer-Implemented Methods

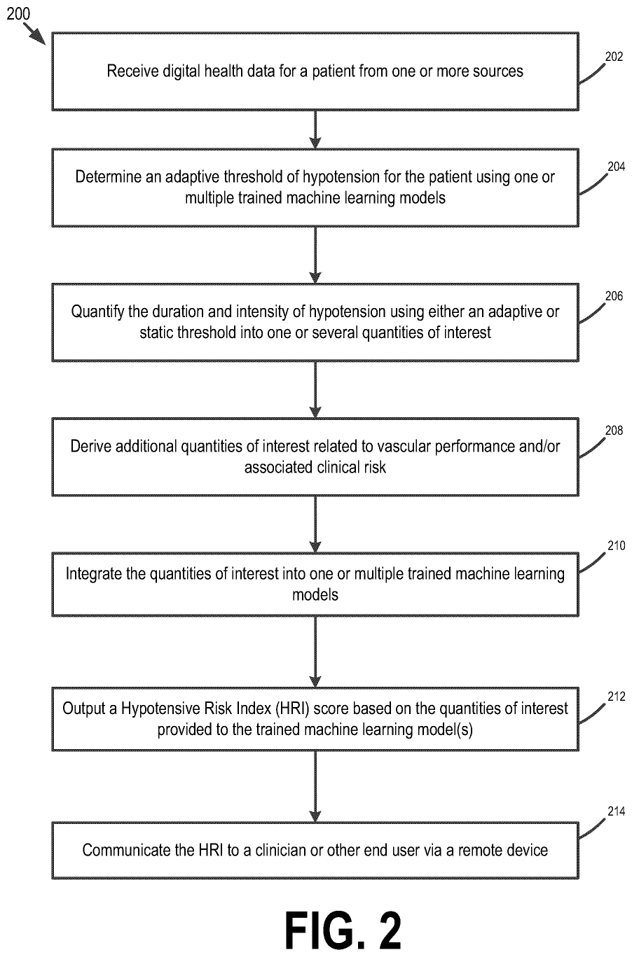

FIG. 2 depicts a computer-implemented method 200 for quantifying and assessing hypotensive risk in patients. This method is designed to leverage digital health data, including blood pressure measurements and other relevant clinical information, to calculate a Hypotensive Risk Index (HRI) score. This score aims to provide healthcare professionals with a comprehensive assessment of a patient's hypotension risk, facilitating more informed clinical decisions.

The method 200 may include receiving digital health data for a patient from one or more sources (block 202). This step involves gathering data from various sources such as electronic health records (EHR), sensors, and monitoring devices. The data acquisition process is critical for ensuring that comprehensive and up-to-date patient information is available for analysis.

The method 200 may include determining an adaptive threshold of hypotension for the patient using one or more trained machine learning models (block 204). This step involves analyzing the received health data to establish a personalized hypotension threshold for the patient, which can vary based on the patient's unique health profile and historical blood pressure data.

The method 200 may include quantifying the duration and intensity of hypotension using either an adaptive or static threshold into one or several quantities of interest (block 206). This quantification process involves calculating metrics such as the hypotension deficit and debt, which represent the severity and cumulative effect of hypotensive episodes over time.

The method 200 may include deriving additional quantities of interest related to vascular performance and/or associated clinical risk (block 208). This involves analyzing various clinical parameters and health data to identify additional factors that may influence the patient's hypotension risk and overall vascular health.

The method 200 may include integrating the quantities of interest into one or more trained machine learning models (block 210). This step leverages advanced analytics and machine learning techniques to synthesize the collected data and quantities of interest, enhancing the accuracy and predictive power of the HRI score.

The method 200 may include outputting a Hypotensive Risk Index (HRI) score based on the quantities of interest provided to the trained machine learning model(s) (block 212). The HRI score is a comprehensive metric that reflects the patient's risk of experiencing hypotension-related complications, enabling clinicians to make more informed treatment decisions.

The method 200 may include communicating the HRI to a clinician or other end user via a remote device (block 214). This step ensures that the calculated HRI score is promptly and securely delivered to healthcare professionals, facilitating timely interventions and patient care.

The method 200 may include receiving blood pressure data from continuous noninvasive blood pressure monitoring devices. This step allows for the continuous monitoring of blood pressure, providing real-time data that can enhance the accuracy of hypotension risk assessment. Continuous monitoring enables the detection of subtle changes in blood pressure patterns, which can be crucial for early intervention and management of hypotension.

The method 200 may include receiving blood pressure data from intermittent noninvasive oscillometric blood pressure monitoring devices. Intermittent monitoring offers snapshots of the patient's blood pressure at specific intervals, contributing to the overall assessment of hypotension risk. This data can be particularly useful for patients who do not require continuous monitoring but still need regular blood pressure assessments.

The method 200 may include receiving blood pressure data from invasive arterial blood catheters. Invasive monitoring provides highly accurate blood pressure readings, which can be essential for critically ill patients or those undergoing complex medical procedures. This data source contributes to a comprehensive understanding of the patient's hemodynamic status and hypotension risk.

The method 200 may include preprocessing the received blood pressure data to standardize data from different sources. Preprocessing ensures that blood pressure readings from various monitoring devices are compatible and can be integrated into the HRI calculation. This step involves converting data into a uniform format, aligning measurement scales, and resolving discrepancies between different data sources. The blood pressure waveform may be preprocessed in the method 200 using source standardization, so that given many sources of BP, any values received by the system 100 are comparable. The method 200 may include an arterial line preprocessing technique including receiving BP components (SBP, DBP, MAP, etc.); optionally receiving other components (HRV, PP), though this could also be described in the derivation of additional metrics. The method 200 may include continuous noninvasive method. In that case, components may be received if it is a waveform, or use as-is if it returns SBP, DBP, etc. In some aspects, the method 200 may preprocess automatic cuff or other periodic measurements by interpolating between measurements. The method 200 may include interpolation of missing segments to determine whether missing values are due to noise or an absence of monitoring.

The method 200 may include interpolating missing segments of blood pressure data. Interpolation addresses gaps in the blood pressure data, ensuring that the HRI calculation is based on a complete and continuous dataset. This technique estimates missing values based on surrounding data points, providing a more accurate representation of the patient's blood pressure profile over time.

The method 200 may include applying a multiple imputation strategy using a Bayesian ridge regression framework to impute missing values based on the patient's present condition. This advanced imputation technique leverages the patient's current health status to predict missing blood pressure values, enhancing the accuracy and reliability of the HRI score. By considering the patient's overall condition, this approach ensures that imputed values are clinically plausible and reflective of the patient's true hemodynamic status.

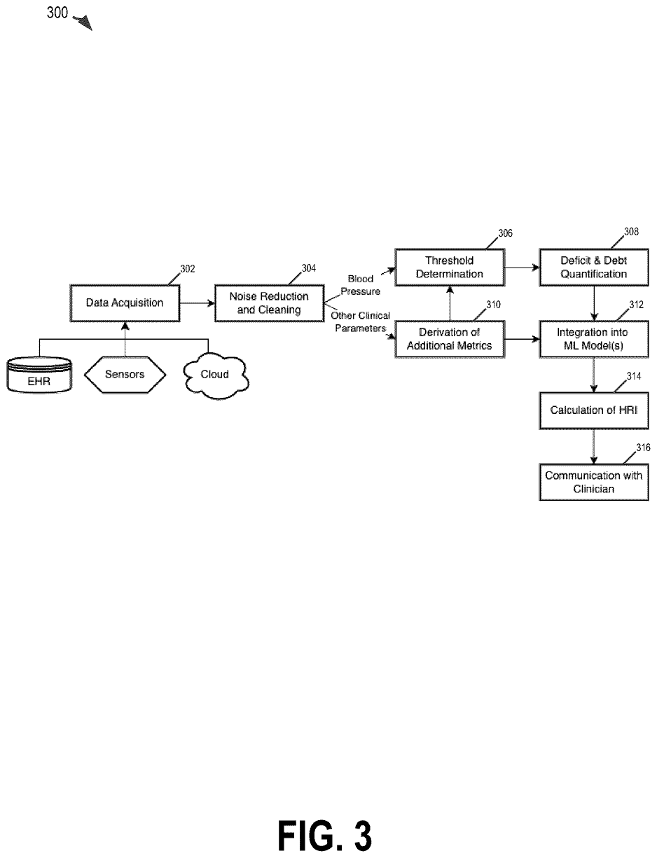

FIG. 3 depicts a flow diagram of a computer-implemented method 300 for quantifying and integrating duration and intensity of hypotension into a Hypotensive Risk Index (HRI), according to some aspects. The method 300 may include acquiring clinical and physiologic data from the electronic health record (EHR), one or more cloud computing resources, one or more individual sensors or monitors, and/or other sources (block 302). These data acquisition sources may correspond to the database 190 depicted in FIG. 1, for example. The method 300 may be carried out by the HRI computing system 104 of FIG. 1, in some aspects.

The acquired clinical and physiologic data may be stored in the memory of one or more processors (e.g., the memory 162 of FIG. 1). The method 300 may include processing the acquired data using one or more processes to clean and reduce signal artifact(s) (block 304). For example, for tabular numeric data (vitals, labs, etc.), the method may include making sure data falls into feasible range (e.g. percents are between 0-100, etc.). The method may standardize units (° F. vs. ° C.). For categorical data (diagnoses, comorbidities, medications, etc.), the method 300 may include mapping values to standardized ontology (ICD10, LOINC, RxNorm). For waveform data (art line, ECG, PPG), the method may include determining a signal quality index.

The acquired data may include blood pressure (BP) and its components (systolic blood pressure [SBP], diastolic blood pressure [DBP], mean arterial pressure [MAP]). The method 300 may include processing some of the acquired data using a threshold determination algorithm, as described herein (block 306). The method 300 may generate an indication of hypotensive deficit when one or more BP values fall below a threshold or score determined by the threshold determination algorithm (block 308), and the accumulation of this deficit over time may be termed hypotension debt.

In some aspects, the method 300 may include parallel computation of additional clinical parameters (block 310) including, but not limited to, electrocardiogram (ECG), photoplethysmography (PPG), vital signs, laboratory values, medical history, and other data collected in the EHR that the method 300 may use either as-is and/or to derive additional metrics such as heart-rate variability (HRV), peripheral oxygen saturation (SpO2), and/or other parameters. The additional data may also be used to help refine the threshold value estimation described herein.

The method 300 may integrate quantities of interest resulting from the hypotension deficit and debt calculations with any additional clinical metrics by processing the quantities through one or machine learning (ML) models trained to predict the likelihood of one or more clinical complications such as organ failure, neurologic injury, death, sepsis, pneumonia, post intensive care syndrome, or others (block 312). The output of these model(s) may be referred to herein as the Hypotension Risk Index (HRI) (block 314). Once calculated, the HRI may be transmitted to clinicians via the electronic health record, cloud, one or more remote devices, and/or other methods (block 316). For example, the HRI may be displayed in a computing device of a clinician in a clinical setting, and/or via a personal device of the clinician.

The method 300 can be implemented in varied ways. The present techniques describe example implementations regarding: 1) details on processing the blood pressure waveform, since that is the major component in terms of processing; 2) structured data such as lab values and vital signs; and 3) example operations of block 310→306.

Processing and filtering blood pressure: arterial blood pressure waveforms are broken into 30-second windows, then adjusted to remove DC offset and correct for slope. The real component of a fast Fourier transform is then calculated, and the resulting spectrum used to measure heart rate over the duration of the window. This heart rate is then used to control the sensitivity when finding systolic peaks in the waveform by adjusting minimum duration between local maxima. Once the sample number of the systolic peak has been determined, this is mapped back to the original, untransformed waveform to yield an instantaneous measure of systolic blood pressure. Additional metrics, such as heart rate variability, can be determined based on the peak-to-peak distances between individual beats.

Structured data: data such as laboratory values and vital signs require less processing, but two important steps include unit validation and conversion (e.g. converting all temperatures in Celsius vs. Fahrenheit, or glucose readings in mg/dL vs. mmol/L. Validation ranges are also used to remove data that fall outside a physiologically reasonable range. This is especially useful to avoid drastic model inputs caused by human error, such as a temperature entry of “371 Celsius” instead of 37.1 Celsius. Additionally, categorical data such as diagnoses, comorbidities, and medications require mapping to standardized ontologies (ICD10, LOINC, RxNorm, etc.).

Box 310 may include the calculation of quantities not directly reported in the source EHR, but which may be important to the HRI model (Box 312) nonetheless. This includes aggregate measures (e.g. anion gap, a measure of electrolyte balance summarized from individual electrolyte levels), existing clinical tools (e.g. SOFA, a composite score used to measure organ dysfunction), and waveform quantities (e.g. SpO2 from a PPG waveform).

These quantities are principally intended for the HRI model (Boxes 312/314), but in some examples an arrow to Box 306 (adaptive threshold determination) allows for the inclusion of oxygenation (and specifically SpO2) in the adaptive thresholding model, as in there may be embodiments that include using oxygenation and perfusion as an addition to the SI-based method above.

### Exemplary Deficit and Debt Quantification

In some aspects, a computer-implemented method for deficit and debt quantification method may include, for an individual patient, computing the hypotension threshold using one or more of the processes above is compared to the patient's current and historical BP measurements.

FIG. 4A depicts a graph 400 of hypothetical patient's SBP over time along with an example threshold of 90 mmHg, according to some aspects. The threshold value is then subtracted from the patient's BP. When the resulting quantity, depicted in FIG. 4B, falls below zero, the patient is said to be experiencing a BP deficit. The magnitude and duration of this deficit may be quantified according to one or more of the following methods:
(i) the proportion of time during the patient's stay in which they were hypotensive (i.e. their BP readings were below the threshold value);
(ii) an integral or summation of the BP deficit over time;
(iii) an exponentially weighted window (EWW) over which the BP deficit is weighted based on how long ago the measurement occurred, then integrated or summarized over time. This approach applies an exponential decay function to the BP measurements, with a half-life set by either the software or the user, which places emphasis on more recent values without letting previous hypotensive episodes fall completely off. The half-life indicates the amount of time at which a historical BP debt measurement is weighted with half the emphasis of the current measurement. The resulting quantity or quantities of interest is termed hypotension debt, as it represents the accumulation of a deficit in BP. FIG. 4C depicts an example of this quantity.

### Exemplary HRI Quantification

As noted, the Hypotension Risk Index (HRI) is a measure of a patient's likelihood of experiencing one or more of the clinical complications associated with hypotension, including but not limited to organ failure, neurologic injury, sepsis, pneumonia, post intensive care syndrome, and/or death. This is accomplished by integrating the measures of BP debt and deficit described above, along with additional clinical parameters, into one or more ML model(s) which are trained to predict the risk of these clinical outcomes.

The additional parameters may include, but are not limited to: physiologic data such as vital signs and laboratory values, contextual information such as medical history or presenting illness, derived quantities such as HRV, or other measurements and quantities relevant to hypotensive risk. The collection of these parameters as well as the BP quantities of interest are termed features or inputs into the HRI model.

The HRI model may include three phases: (i) Imputation; (ii) Prediction; and (iii) Explanation, each of which will now be described in turn.

### Imputation

When considering clinical and physiologic data, missing data elements (that is, elements which have not been measured or recorded for an individual patient) are typically not at random. Clinicians order tests or take measurements when they believe the patient is at risk of a given condition; to collect every test and every measurement on every patient at every time point would be both wasteful and provide little clinical value. Left unchecked, a predictive model can learn these patterns and make inferences about a patient's risk based on the presence or absence of a given measurement, rather than the value of the measurement itself. However, these patterns are subject to frequent change with constantly evolving clinical practice, care guidelines, reimbursement models, and hospital cultures. Thus, the present techniques advantageously improve upon conventional techniques by obfuscating these patterns from the prediction model, such that it learns to rely on the measurement itself rather than its mere presence or absence.

In some aspects, missing elements be mitigated using an imputation technique to fill in missing values. One strategy, when faced with a missing value, is to assume that the missing value is normal. Either the mean or median of the feature in a retrospective training set may be used for this purpose. Another strategy is to isolate the static value using a complex nonlinear model static value, and learn the missingness patterns anyway. More complex imputation may include randomly sampling missing values from a learned distribution based on the training dataset, however, this still relies on the assumption that missing values are in a normal “healthy” range.

The present techniques may include a multiple imputation strategy that uses a Bayesian ridge regression framework to impute missing values by sampling from a learned distribution informed by the patient's present condition. Alternately stated, the information that is known about a given patient is used to inform the distribution of the unknown features, from which a random value is sampled. As an example: a patient has not yet had a complete blood count (CBC), but does have a high temperature, high heart rate, and positive blood culture. Rather than assuming their white blood cell count (WBC) is normal, the distribution of likely WBC values can be informed by these known features. In this case, it is likely that the WBC count is higher than normal. This imputation technique may be repeated for all missing features from an individual patient. The careful imputation of these missing features ensures that the predictive model does not learn unstable and variable patterns in missingness, making it robust to change. Its passivity also ensures that clinicians do not need to change their practice patterns to accommodate the model.

### Prediction

A second step in the calculation of the HRI may be passing the quantities of interest from both the BP debt calculations and the additional imputed clinical and physiologic data described previously into one or more machine learning models trained to predict the risk of one or more adverse events, including but not limited to physiologic outcomes such as renal, hepatic, or neurologic injury; and/or clinical outcomes such as mortality, Intensive Care Unit (ICU) admission or duration, length of stay, or (re) admission to the hospital.

In one aspect, the one or more machine learning models may be architected as a Long Short-Term Memory (LSTM) model, a type of Recurrent Neural Network (RNN) capable of ingesting time-series data. In other aspects, different model types may be used (e.g., a language model). In the example of an RNN, the model may take as input an array containing the value of each feature (the BP Debt, vital signs, laboratory values, and other clinical parameters described previously) as has changed over time during the patient's recent stay. The model may then be trained on historical, retrospective patient data to predict the likelihood of one or more of the adverse physiologic or clinical outcomes described previously. The output of this model is the likelihood of a patient experiencing one or more of the outcomes of interest, expressed either as a calibrated probability (i.e. the percent chance a patient has of experiencing the outcome), as a fold-change in risk (i.e. a given patient is X times more likely to experience a poor outcome than average) and/or as a scaled value not tied to an exact probability. In any of the above cases, higher scores indicate that the patient is more likely to experience an adverse event.

### Explanation

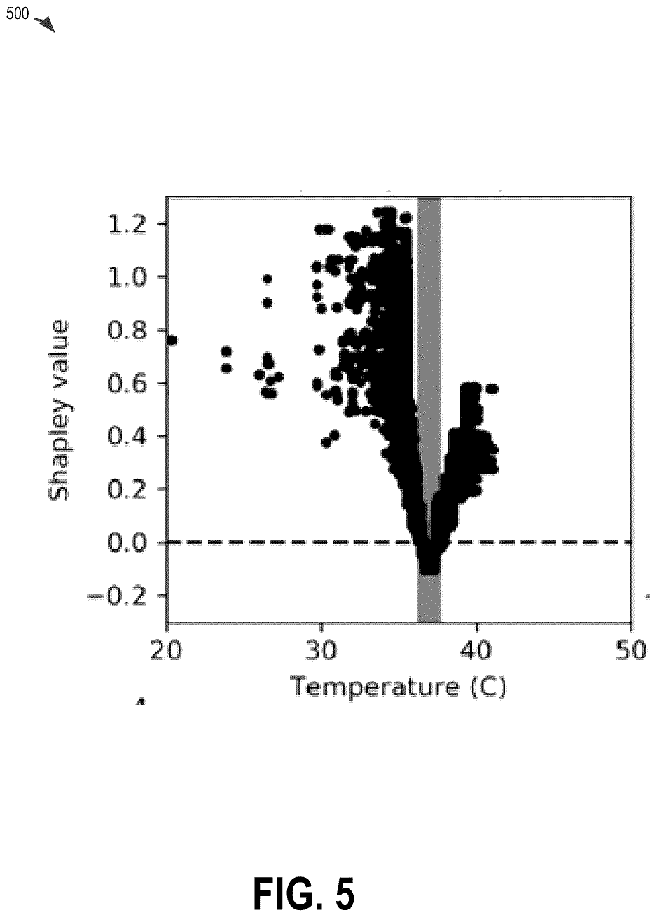

When clinicians interpret a risk score such as the HRI, it is often helpful for them to understand the principal drivers behind the score, (e.g., what is causing the score to be elevated or depressed). Some aspects of the HRI may include the capability to weigh the individual factors used in a prediction and return the most influential variables alongside the HRI score itself. In some aspects, this influence analysis may use a perturbation-based approach such as Shapley values, as depicted in FIG. 5. Shapley values leverage economic game theory to treat a given machine learning model as a value function, distributing surplus predictive value to individual players (features). Thus, for any given prediction on an individual subject, Shapley values represent a principled way to investigate the contributions of each feature to a given prediction.

In practice, this means that in a given prediction on an individual patient, each feature in the model (BP Debt quantities of interest, vital signs, laboratory values, etc.) is assigned an importance score, with positive values indicating that the variable pushed the model toward predicting higher HRI scores (more likely to experience an adverse event), and negative predictive values pushing the model towards lower scores (less likely to predict an adverse event).

This can be used in two ways: first, the Shapley values may be aggregated over all predictions in a dataset to provide insight into which features are generally most impactful. FIG. 5 specifically depicts an example of one embodiment of this summary view 500, where the magnitude of the Shapley value is plotted against the feature value itself. In this case, the model has learned temperatures around 37-38° C. (normal human body temperature) do not contribute to the risk of deterioration, while large deviations (either negative (hypothermia), or positive (fever) tend to increase risk. This aggregation analysis can be performed on all features in the dataset, providing an at-a-glance view of how the model is using each one of its inputs.

Second, Shapley values may be used at an individual patient-level to provide clinicians with context surrounding their patient. For example, the clinician may quickly adjudicate which factors are causing an increase in score. This has two principal advantages: first, it allows the clinician to identify which factors are most important to the patient's management, and make care decisions accordingly. Second, it allows clinicians to rule out factors with a known cause. For example, the model may indicate that a patient's low white blood cell count is of concern, but if the clinician knows that the patient is receiving chemotherapy, it may still be tolerable.

Thus, as shown, explanations may be achieved through various methods, including for a given prediction on an individual patient, input data is ingested by the model, and the HRI score is determined. Subsequently, the same input data is fed back into the model with small tweaks made to individual features. The updated model score is then noted. Features that, when altered, yield large changes in the model score are deemed more important than features that do not impact the model. From there explanations of the scoring can be provided.

### Example Machine Learning Training

We now describe a prophetic example training for a full adaptive threshold of hypotension and HRI models.

Cohort: In the prophetic example, the cohort would include a population of ICU patients at Michigan Medicine from 2014-2024, aged ≥18 years, and who had both an intra-arterial catheter with pressure transduction and photoplethysmography (PPG) monitored simultaneously for at least 24 hours.

Data from 2014-2021 would be used variously for training and validation, and the years 2022-2024 preserved as a hold-out test set. Subjects in the 2014-2021 data were segmented by hospital encounter, and randomly assigned to one of three groups: training for the adaptive threshold model (30%), training for the HRI model (50%), and a validation set for model selection and hyperparameter tuning (20%). Subjects with hospital encounters occurring in these training and validation sets were removed from the hold out test set, so as to prevent the model from learning patient-specific patterns.

Adaptive hypotension threshold model: The threshold of what constitutes severe or damaging hypotension can vary between patients based on their clinical status and individual compensatory reserve. One method by which the heart continually balances blood pressure is by varying Heart Rate (HR; number of beats per minute) and Stroke Volume (SV; the volume of blood pumped per beat). The product of these two quantities is termed Cardiac Output (CO), and represents the volume of blood pumped by the heart per minute. Typically, this exists in a dynamic equilibrium: if blood pressure is low, the heart will increase CO to increase it. If blood pressure is high, the heart backs off and lowers CO, all else being equal.

However, in severe cases of hypotension, the heart cannot increase its CO high enough to compensate for this drop in pressure. This is often simplified as Shock Index (SI), a quantity defined as HR divided by systolic blood pressure (SBP). Values greater than 1 (i.e. HR>SBP) indicate the heart may be struggling to maintain the current blood pressure, and these patients have demonstrably greater morbidity and mortality [1,2].

Taken together, we would train the threshold of hypotension model, based on shock index, that can be used to estimate the threshold of dangerous hypotension for an individual patient. Three quantities of interest are calculated or estimated from the arterial pressure transducer: SBP, HR, and SV (the latter two are then used to calculate CO). As the patient is monitored at baseline, transient decreases in SBP are met by corresponding increases in CO, and vice versa. However, if a decrease in SBP is instead met by a decrease in CO, it indicates that this may be a pressure that is not sustainable for the patient. Mathematically, this can be represented by a moving window Pearson correlation. The resulting quantity ranges between [−1, 1]; values >0 indicate that decreases in BP are not being met with corresponding decreases in CO, and that the patient may be struggling to compensate. The BP levels at which this positive correlation occurs, then represent a candidate hypotension threshold for the purposes of quantifying hypotension debt.

The adaptive threshold training cohort (30%) indicated above will be used to tune the parameters of this model. This includes the correlation window size, cutoff threshold and duration for labeling episodes of decompensation.

HRI Score Model: After hypotension debt has been calculated based on either a set threshold, or the adaptive one discussed above, the debt measurement can be integrated with other clinical parameters to create an overall picture of risk. The ability to include debt/deficit measurements and optionally more contextual data (e.g., diagnoses, comorbidities, medical history) provides numerous advantages over the prior art. On the 50% training cohort delineated above, the input data would be collected from the retrospective data warehouses (such as, Clarity and/or Epic Systems). This includes vital signs (respiratory rate, heart rate, blood pressure, temperature, Glasgow coma score, etc.), laboratory values (electrolytes, hematology, coagulation metrics, metabolites [lactate/glucose], etc.), and other clinical contextual data (diagnosis, patient service, procedure history, active medications, etc.). This input data is then structured to simulate individual observations made every 15 minutes using the most up-to-date data available at that time. Missing data would then be imputed using the strategy described in the spec sheet.

Outcome labels are then assigned to the individual observations depending on the outcome of the patient. These may include the onset of organ injury (renal, myocardial, cerebral, etc.) or clinical markers of deterioration (unexpected ICU transfer, mechanical ventilator use, cardiac and respiratory arrest, and/or mortality). Relevant outcome labels will then be collated into a single composite prediction target, both on the encounter level (did this event happen during the patient's hospital stay) or on the observation level (did this event happen in the e.g. 24 hours following the individual observation).

The model will be evaluated using both threshold-independent metrics (Receiver-Operator Characteristic Curve, Precision-Recall Curve), as well as binary metrics calculated at a variety of precision levels: positive predictive value, negative predictive value, sensitivity, specificity, and F1 score.

It will be appreciated by persons skilled in the art that the present techniques provide numerous advantages over conventional systems and may be implemented in various ways.

In various implementations of the present techniques, three types of inputs relevant to HRI (structured numeric, structured categorical, and unstructured waveforms) may be processed differently in different examples.

The structured numeric data may include things like vitals and labs, which occur at irregular intervals repeatedly throughout the patient's stay. For training, two things may be regularized: timing and missingness. For timing, we can simulate the model running every e.g. 15 m by defining those time intervals a priori, and then looking backwards and using the most recently-available entry at each timestep, for each patient. For missingness, we can perform imputation so that the model doesn't learn volatile clinician behavior, and can focus on physiology. The “Imputation” section in the original spec sheet goes into more detail here.

Structured categorical data is similar, but is mapped to discrete, defined categories to be useful to a model. For instance, a human may read “vomiting” and “throwing up” as the same, but these have to be mapped to a standardized ontology.

Unstructured waveform data is to be structured in various examples. This may be done by breaking into usable chunks (here, 30 s intervals), and applying the processing steps as in “processing and filtering blood pressure” above. For arterial blood pressure waveforms, the output of these chunks (SBP, HR, SV, CO) are then aggregated in differing amounts to create moving windows of variable size of the patient's stay for the Pearson correlation as described above. Additionally, pulse pressure (SBP-DBP) and respiratory sinus arrhythmia (changes in heart rate and blood pressure due to breathing) can be used as approximations of volume status when the full ABP waveform is not present.

Further, the trained models herein may be trained to produce different types of outputs, in various implementations of the present techniques.

In various examples, we use a composite outcome to create a probabilistic binary prediction, i.e., what is the likelihood of any one of the following events occurring. However, this can also be expressed as probabilities over multiple timepoints, e.g. what is the likelihood of the event occurring in the next 24 hours vs. during the entire encounter vs. within 30 days of discharge.

There are also models that output ordinal (0=healthy, 1=low-level injury, 2=medium-level injury, 3=high level injury) or multiple outcomes (0=healthy, 1=kidney injury, 2=heart injury, 3=death), but there are mathematical complications with these models that make them less interpretable, and in our experience, less useful. It is often advantageous to train differing models on the same data to predict different outcomes. If it is clinically useful to differentiate between outcomes, it is often better to train multiple models in an ensemble, all using different targets. That way, a patient's risk of kidney injury is assessed and reported independently of their risk of cardiac injury, for example.

Further still, in various implementations, the HRI score explanation may be achieved in different ways with different machine learning architectures.

In simpler models (e.g. logistic regression, LASSO, etc.) coefficient weights are often used to assess a feature's importance in the overall model, and then these coefficient weights can be multiplied by the inputs to understand the importance in an individual prediction. However, for more complex models with structured input data, SHAP can be implemented as the gold-standard when it comes to explainability. It has the additional benefit of being model agnostic, meaning that while it is well optimized to work with specific architectures (gradient boosting trees being a major one), in theory it will work with any model type.

Further still, in various implementations, the machine learning architectures herein may be modified. For example, the addition of real-time waveform processing may be used. The addition of clinical contextual data, for example diagnoses, comorbidities, procedure history, and active medications can be used. These are actively excluded from prior art techniques to reduce redundancy to clinicians. However, many medications or diagnoses impact acceptable hypotension thresholds (e.g. it may be more acceptable for a patient with congestive heart failure to experience transient hypotension in an effort to lower their blood pressure, when compared to a patient at risk of hypovolemic shock).

With the present techniques we able to identify previously-unknown associations with different metrics that classify HRI, including in addition to organ injury (kidney, liver, myocardial), possible previously-unknown associations include ARDS, cognitive deficits/worsening TBI, intestinal ischemia, myocardial infarction, and perhaps pulmonary embolism and DVT.

### Aspects

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

Aspects of the techniques described in the present disclosure may include any of the following aspects, either alone or in combination:

1. A computing system comprising: a processor and a memory having stored thereon computer-executable instructions that, when executed, cause the computing system to receive digital health data for a patient from one or more sources; determine an adaptive threshold of hypotension for the patient using one or more trained machine learning models; quantify the duration and intensity of hypotension using either an adaptive or static threshold into one or several quantities of interest; derive additional quantities of interest related to vascular performance and/or associated clinical risk; integrate the quantities of interest into one or more trained machine learning models; output a Hypotensive Risk Index (HRI) score based on the quantities of interest provided to the trained machine learning model(s); and communicate the HRI to a clinician or other end user via a remote device.

2. The computing system of aspect 1, further comprising receiving blood pressure data from continuous noninvasive blood pressure monitoring devices or receiving blood pressure derived data.

3. The computing system of any of aspects 1-2, further comprising receiving blood pressure data from intermittent noninvasive oscillometric blood pressure monitoring devices.

4. The computing system of any of aspects 1-3, further comprising receiving blood pressure data from invasive arterial blood catheters.

5. The computing system of any of aspects 1-4, further comprising preprocessing the received blood pressure data to standardize data from different sources.

6. The computing system of any of aspects 1-5, further comprising interpolating missing segments of blood pressure data.

7. The computing system of any of aspects 1-6, further comprising applying a multiple imputation strategy using a Bayesian ridge regression framework to impute missing values based on the patient's present condition.

8. The computing system of any of aspects 1-7, wherein the quantities of interest are integrated into one or more trained machine learning models trained to predict a likelihood of one or more of organ failure, neurologic injury, sepsis, pneumonia, post intensive care syndrome, and death.

9. A computer-implemented method comprising: receiving, via one or more processors, digital health data for a patient from one or more sources; determining, via one or more processors, an adaptive threshold of hypotension for the patient using one or more trained machine learning models; quantifying, via one or more processors, the duration and intensity of hypotension using either an adaptive or static threshold into one or several quantities of interest; deriving, via one or more processors, additional quantities of interest related to vascular performance and/or associated clinical risk; integrating, via one or more processors, the quantities of interest into one or more trained machine learning models; outputting, via one or more processors, a Hypotensive Risk Index (HRI) score based on the quantities of interest provided to the trained machine learning model(s); and communicating, via one or more processors, the HRI to a clinician or other end user via a remote device.

10. The method of aspect 9, further comprising receiving blood pressure data from continuous noninvasive blood pressure monitoring devices or receiving blood pressure derived data.

11. The method of any of aspects 9-10, further comprising receiving blood pressure data from intermittent noninvasive oscillometric blood pressure monitoring devices.

12. The method of any of aspects 9-11, further comprising receiving blood pressure data from invasive arterial blood catheters.

13. The method of any of aspects 9-12, further comprising preprocessing the received blood pressure data to standardize data from different sources.

14. The method of any of aspects 9-13, further comprising interpolating missing segments of blood pressure data.

15. The method of any of aspects 9-14, further comprising applying a multiple imputation strategy using a Bayesian ridge regression framework to impute missing values based on the patient's present condition.

16. The method of any of the aspects claim 9-15, wherein integrating the quantities of interest into the one or more trained machine learning models comprises integrating the quantities of interest into the one or more trained machine learning models trained to predict a likelihood of one or more of organ failure, neurologic injury, sepsis, pneumonia, post intensive care syndrome, and death.

17. A computer-readable medium having stored thereon instructions that when executed cause a computer to perform steps comprising: receiving digital health data for a patient from one or more sources; determining an adaptive threshold of hypotension for the patient using one or more trained machine learning models; quantifying the duration and intensity of hypotension using either an adaptive or static threshold into one or several quantities of interest; deriving additional quantities of interest related to vascular performance and/or associated clinical risk; integrating the quantities of interest into one or more trained machine learning models; outputting a Hypotensive Risk Index (HRI) score based on the quantities of interest provided to the trained machine learning model(s); and communicating the HRI to a clinician or other end user via a remote device.

18. The computer-readable medium of aspect 17, further comprising instructions for receiving blood pressure data from continuous noninvasive blood pressure monitoring devices or for receiving blood pressure derived data.

19. The computer-readable medium of any of aspects 17-18, further comprising instructions for receiving blood pressure data from intermittent noninvasive oscillometric blood pressure monitoring devices.

20. The computer-readable medium of any of aspects 17-19, further comprising instructions for receiving blood pressure data from invasive arterial blood catheters.

21. The computer-readable medium of any of aspects 17-20, further comprising instructions for preprocessing the received blood pressure data to standardize data from different sources.

22. The computer-readable medium of any of aspects 17-21, further comprising instructions for applying a multiple imputation strategy using a Bayesian ridge regression framework to impute missing values based on the patient's present condition.

23. The computer-readable medium of any of aspects 1722, where the instructions for integrating the quantities of interest into the one or more trained machine learning models comprise instructions for integrating the quantities of interest into the one or more trained machine learning models trained to predict a likelihood of one or more of organ failure, neurologic injury, sepsis, pneumonia, post intensive care syndrome, and death.

### ADDITIONAL CONSIDERATIONS

The following considerations also apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

It should also be understood that, unless a term is expressly defined in this patent using the sentence “As used herein, the term” “is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112 (f).

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of “a” or “an” is employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for implementing the concepts disclosed herein, through the principles disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A computing system comprising: a processor and a memory having stored thereon computer-executable instructions that, when executed, cause the computing system to:
receive digital health data for a patient from one or more sources;
determine an adaptive threshold of hypotension for the patient using one or more trained machine learning models;
quantify the duration and intensity of hypotension using either an adaptive or static threshold into one or several quantities of interest;
derive additional quantities of interest related to vascular performance and/or associated clinical risk;
integrate the quantities of interest into one or more trained machine learning models;
output a Hypotensive Risk Index (HRI) score based on the quantities of interest provided to the trained machine learning model(s); and
communicate the HRI to a clinician or other end user via a remote device.

2. The computing system of claim 1, the memory having stored thereon instructions that when executed, cause the computing system to:
receive blood pressure data from continuous noninvasive blood pressure monitoring devices or receiving blood pressure derived data.

3. The computing system of claim 1, the memory having stored thereon instructions that when executed, cause the computing system to:
receive blood pressure data from intermittent noninvasive oscillometric blood pressure monitoring devices.

4. The computing system of claim 1, the memory having stored thereon instructions that when executed, cause the computing system to:
receive blood pressure data from invasive arterial blood catheters.

5. The computing system of claim 1, the memory having stored thereon instructions that when executed, cause the computing system to:
preprocess the received blood pressure data to standardize data from different sources.

6. The computing system of claim 1, the memory having stored thereon instructions that when executed, cause the computing system to:
interpolate missing segments of blood pressure data.

7. The computing system of claim 1, further comprising applying a multiple imputation strategy using a Bayesian ridge regression framework to impute missing values based on the patient's present condition.

8. The computing system of claim 1, wherein the quantities of interest are integrated into one or more trained machine learning models trained to predict a likelihood of one or more of organ failure, neurologic injury, sepsis, pneumonia, post intensive care syndrome, and death.

9. A computer-implemented method, comprising:
receiving, via one or more processors, digital health data for a patient from one or more sources;
determining, via one or more processors, an adaptive threshold of hypotension for the patient using one or more trained machine learning models;
quantifying, via one or more processors, the duration and intensity of hypotension using either an adaptive or static threshold into one or several quantities of interest;
deriving, via one or more processors, additional quantities of interest related to vascular performance and/or associated clinical risk;
integrating, via one or more processors, the quantities of interest into one or more trained machine learning models;
outputting, via one or more processors, a Hypotensive Risk Index (HRI) score based on the quantities of interest provided to the trained machine learning model(s); and
communicating, via one or more processors, the HRI to a clinician or other end user via a remote device.

10. The method of claim 9, further comprising receiving blood pressure data from continuous noninvasive blood pressure monitoring devices or receiving blood pressure derived data.

11. The method of claim 9, further comprising receiving blood pressure data from intermittent noninvasive oscillometric blood pressure monitoring devices.

12. The method of claim 9, further comprising receiving blood pressure data from invasive arterial blood catheters.

13. The method of claim 9, further comprising preprocessing the received blood pressure data to standardize data from different sources.

14. The method of claim 9, further comprising interpolating missing segments of blood pressure data.

15. The method of claim 9, further comprising applying a multiple imputation strategy using a Bayesian ridge regression framework to impute missing values based on the patient's present condition.

16. The method of claim 9, wherein integrating the quantities of interest into the one or more trained machine learning models comprises integrating the quantities of interest into the one or more trained machine learning models trained to predict a likelihood of one or more of organ failure, neurologic injury, sepsis, pneumonia, post intensive care syndrome, and death.

17. A non-transitory computer-readable medium having stored thereon instructions that when executed cause a computer to perform steps comprising:
receiving digital health data for a patient from one or more sources;
determining an adaptive threshold of hypotension for the patient using one or more trained machine learning models;
quantifying the duration and intensity of hypotension using either an adaptive or static threshold into one or several quantities of interest;
deriving additional quantities of interest related to vascular performance and/or associated clinical risk;
integrating the quantities of interest into one or more trained machine learning models;
outputting a Hypotensive Risk Index (HRI) score based on the quantities of interest provided to the trained machine learning model(s); and
communicating the HRI to a clinician or other end user via a remote device.

18. The computer-readable medium of claim 17, further comprising instructions for receiving blood pressure data from continuous noninvasive blood pressure monitoring devices or for receiving blood pressure derived data.

19. The computer-readable medium of claim 17, further comprising instructions for receiving blood pressure data from intermittent noninvasive oscillometric blood pressure monitoring devices.

20. The computer-readable medium of claim 17, further comprising instructions for receiving blood pressure data from invasive arterial blood catheters.

21. The computer-readable medium of claim 17, further comprising instructions for preprocessing the received blood pressure data to standardize data from different sources.

22. The computer-readable medium of claim 17, further comprising instructions for applying a multiple imputation strategy using a Bayesian ridge regression framework to impute missing values based on the patient's present condition.

23. The computer-readable medium of claim 17, where the instructions for integrating the quantities of interest into the one or more trained machine learning models comprise instructions for integrating the quantities of interest into the one or more trained machine learning models trained to predict a likelihood of one or more of organ failure, neurologic injury, sepsis, pneumonia, post intensive care syndrome, and death.
